(19) United States
(12) Patent Application Publication
(10) Pub. No.: US 2015/0012047 A1
(43) Pub. Date: Jan. 8, 2015

(54) Medical Equipment for Anchoring and Closing of Cranial Flap during Neurosurgery of Head

(71) Applicant: Ghansham Das AGARWAL, (US)

(72) Inventor: Ghansham D. Agarwal, Kolkata (IN)

(21) Appl. No.: 14/372,428

(22) PCT Filed: Apr. 30, 2013
(86) PCT No.: PCT/IN2013/000289

§ 371 Date: Jul. 15, 2014

(30) Foreign Application Priority Data
Dec. 19, 2012 (IN) 1431/KOL/2012

(51) Int. Cl. A61B 17/68; A61B 17/80

(52) U.S. Cl. CPC A61B 17/688; A61B 17/8061
USPC 606/286

(57) ABSTRACT

This invention relates to a medical equipment for anchoring and closing of cranial flap during neurosurgery of head comprising of a plurality of arms including at least one longer arm and several smaller arms made of biocompatible material.

### FIELD OF INVENTION

This invention relates to a medical equipment for anchoring and closing of cranial flap during neurosurgery of head.

### BACKGROUND/PRIOR ART OF INVENTION

Surgical operations inside the head/on the brain are performed after making an opening in the skull i.e. the bony cage around the brain or cranium. Generally adopted method of opening is to create four/three burr holes (holes are made with the help of burrs followed by cutting in between the holes by means of giggly saw or latest osteotomes (powered tools). The hard shell flap/bony flap/cranial flap thus created is either kept attached on one side with the pericranium or kept away from patient by covering in a moist mop or in saline.

After finishing of operation on brain, this flap needs to be reapplied and attached with the skull. There are different methods to attach it.

Conventional method comprises steps of drilling small holes in skull followed by inserting stainless steel wire through these holes. Thereafter, the wire is twisted, cut to size and bent inside.

Another method makes utilization of two absorbable plastic buttons, one under the skull and one above the skull. Both buttons are tied with absorbable thread. This keeps the flap in position.

Still another method comprises application of two metal/titanium discs. One disc which goes under the skull has a serrated central wire, wherein the serrations are directed downwards. The second disc is threaded on the serrated wire, which has a central hole with locking fins and can be moved downwards only. It requires special instruments to pull the wire through second disc, in which excess wire is cut off and second disc is locked on the skull.

### Disadvantages:

It has poor stability and grip. There are chances of flap slipping down. The buttons are quite thick and can be felt under skin and sometimes may look ugly if applied on exposed surface of head (forehead etc). Application of these buttons is also time consuming. Further, these are expensive.
It provides good anchorage. However, it requires special & expensive instruments. At times, reoperation is required due to complication or other reason, wherein reoperation is cumbersome and also requires special tools. It is also time consuming and very expensive.

Hence, it can be concluded that, the aforesaid prior arts provide only limited stability to cranial flap. Slight push/pressure can push in the flap and damage brain. Therefore, it is required to develop a medical equipment for anchoring and closing of cranial flap during neurosurgery of head, which can address the above drawbacks.

### OBJECTS OF INVENTION

An object of this invention is to propose a medical equipment for anchoring and closing of cranial flap during neurosurgery of head.

Another object of this invention is to propose a medical equipment for anchoring and closing of cranial flap during neurosurgery of head which is very fast to use and apply (less than two minutes).

Still another object of this invention is to propose a medical equipment for anchoring and closing of cranial flap during neurosurgery of head which is cost effective.

Still another object of this invention is to propose a medical equipment for anchoring and closing of cranial flap during neurosurgery of head which does not need any special instrumentation for application.

Still another object of this invention is to propose a medical equipment for anchoring and closing of cranial flap during neurosurgery of head which is robust enough to resist pressure to prevent damage to brain.

Still another object of this invention is to propose a medical equipment for anchoring and closing of cranial flap during neurosurgery of head wherein re-opening of craniotomy flap is fast without requiring special instrumentation.

Still another object of this invention is to propose a medical equipment for anchoring and closing of cranial flap during neurosurgery of head which is reusable.

Still another object of this invention is to propose a medical equipment for anchoring and closing of cranial flap during neurosurgery of head which allows outward movement of cranial flap in case of brain edema.

### STATEMENT OF INVENTION

According to this invention, there is provided a medical equipment for anchoring and closing of cranial flap during neurosurgery of head comprising of a plurality of arms including at least one longer arm and several smaller arms made of biocompatible material.

### BRIEF DESCRIPTION OF THE ACCOMPANYING DRAWINGS

Further objects and advantages of this invention will be more apparent from the ensuing description when read in conjunction with the accompanying drawings and wherein:

FIG. 1 shows: top view of embodiment.

FIG. 2 shows: side view of first embodiment.

FIG. 3 shows: application of first embodiment on cranium from top.

FIG. 4 shows: top view of the embodiment.

FIG. 5 shows: bottom view of the embodiment.

FIG. 6 shows: applied on skull top view of the embodiment.

FIG. 7 shows: bottom view of the embodiment.

FIG. 8 shows: top view of the embodiment.

FIG. 9 shows: applied on skull top.

FIG. 10 shows: applied on skull inside view.

FIG. 11 shows: bottom view of the embodiment.

FIG. 12 shows: applied on skull.

FIGS. 13 & 14 shows: applied inside skull view.

### DETAILED DESCRIPTION OF THE INVENTION WITH REFERENCE TO THE ACCOMPANYING DRAWINGS

The present invention discloses a medical equipment for anchoring and closing of cranial flap during neurosurgery of head. Said device comprising of a plurality of arms constituting octopus shaped metallic sheet. It is made of biocompatible material such as Titanium.

The instant invention comprising of several exemplary embodiments discussed herein below:

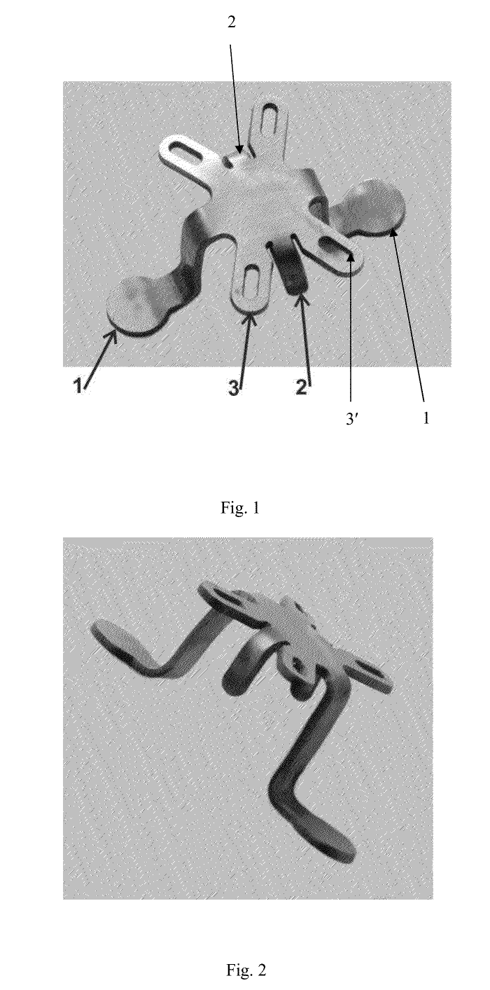

Now, reference may be made to FIG. 1, in which the device comprising of two longer arms (1) opposite to each other and bent in “Z” shape. Further, two more arms (2) are provided opposite to each other, which are comparatively smaller constituting L-section when viewed from side as shown in FIG. 2. Again, four cross arms (3) of same size with hole (3′) are disposed between the longer and smaller arms (1 and 2).

Thus, the Z-shaped bent arms (1) anchor under the skull to hold flap in position and four cross arms (3) anchor above skull. Two small arms (2) prevent side movements. This embodiment is shown in FIG. 3 during operation on head.

It is very fast and easy to use. Further, this does not need any instrumentation.

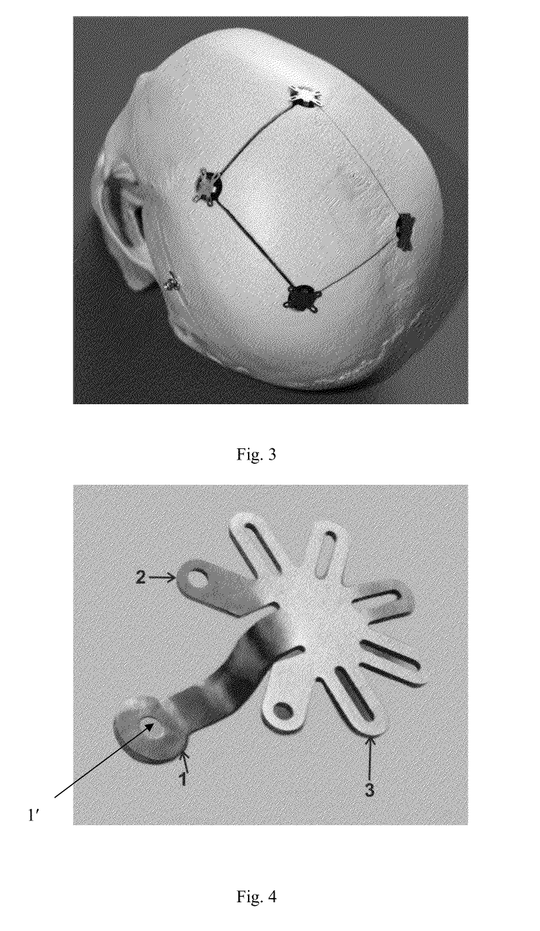

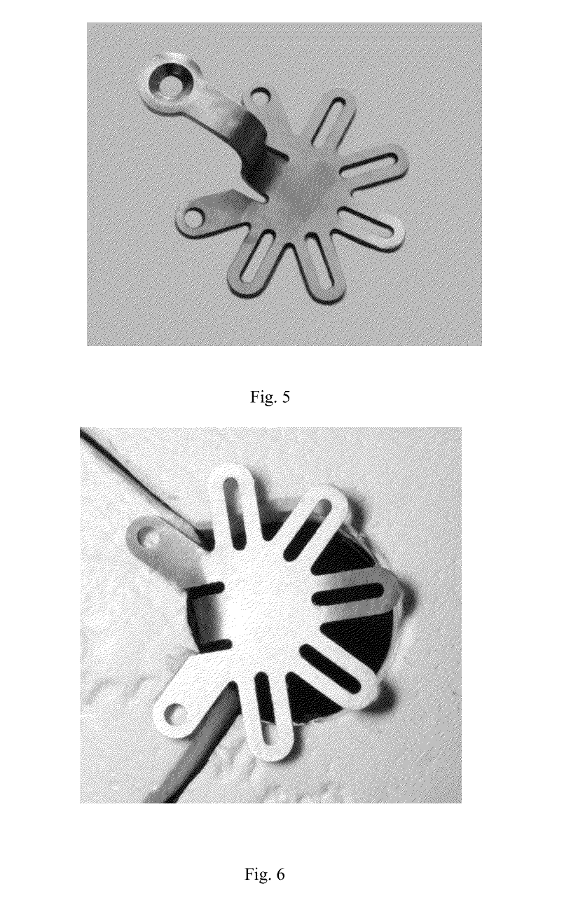

Now, reference may be made to FIGS. 4 and 5, wherein another embodiment of the equipment comprising of a long arm (1), which is bent in Z-shape with a hole (1′) for application of screw there through. Screwing requires a screw and a screw driver.

It further comprises a plurality of smaller arms (2 & 3) with hole integrated to the longer arm (1) as shown.

Referring to FIG. 6, which indicates application of the above embodiment on skull during operation. During operation, smaller arms are positioned on the skull and longer arm is screwed to the under skull.

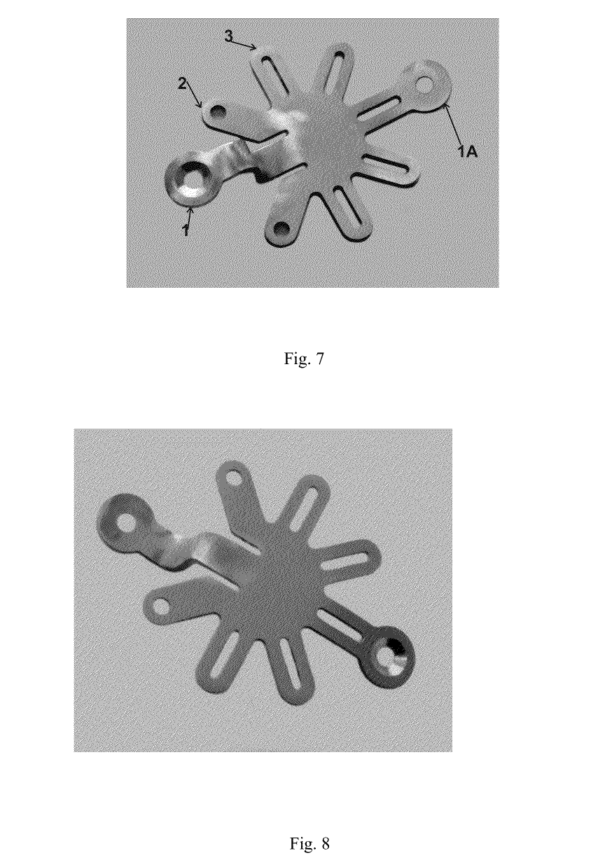

Again, reference may be made to FIGS. 7 and 8, indicating still another embodiment comprising of two long arms (1 and 1A) disposed opposite to each other with a hole, wherein one arm (1) is bent and other arm (1A) is straight. A plurality of smaller arms (2 and 3) with hole are disposed between the longer arms.

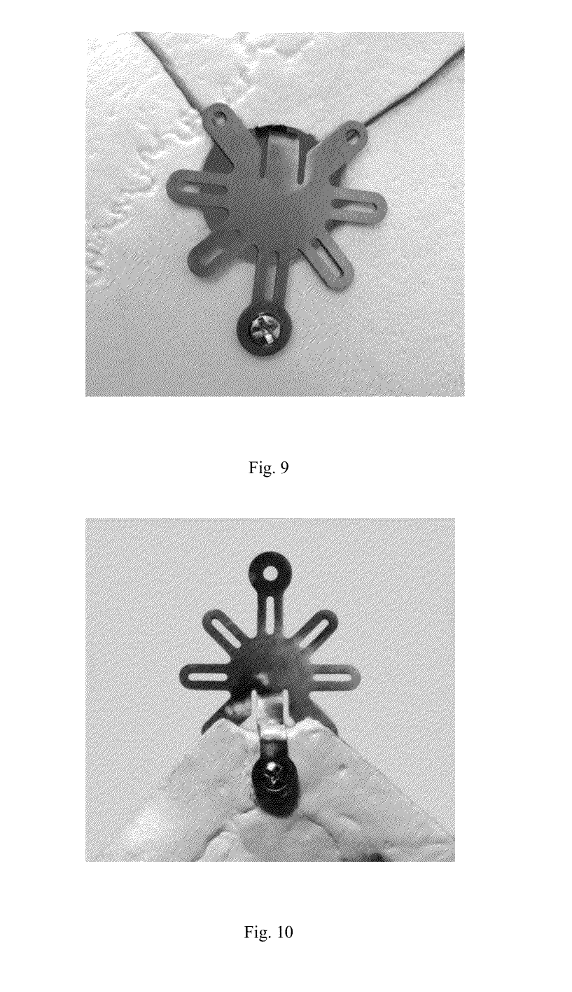

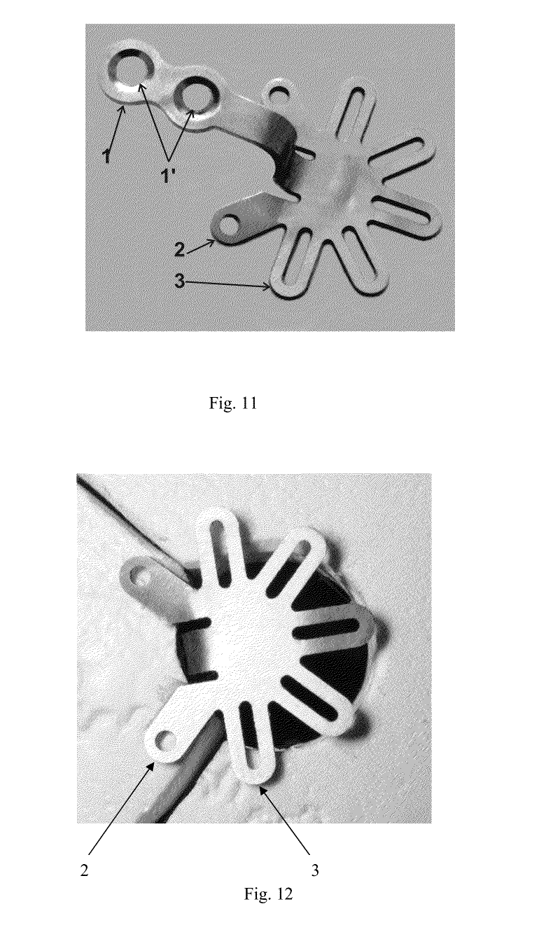

Reference may be made to FIGS. 9 and 10 indicating application of the device on skull top and inside wherein two screws are required. Out of the two screws, one screw is applied inside the flap and other one is on the top of skull.

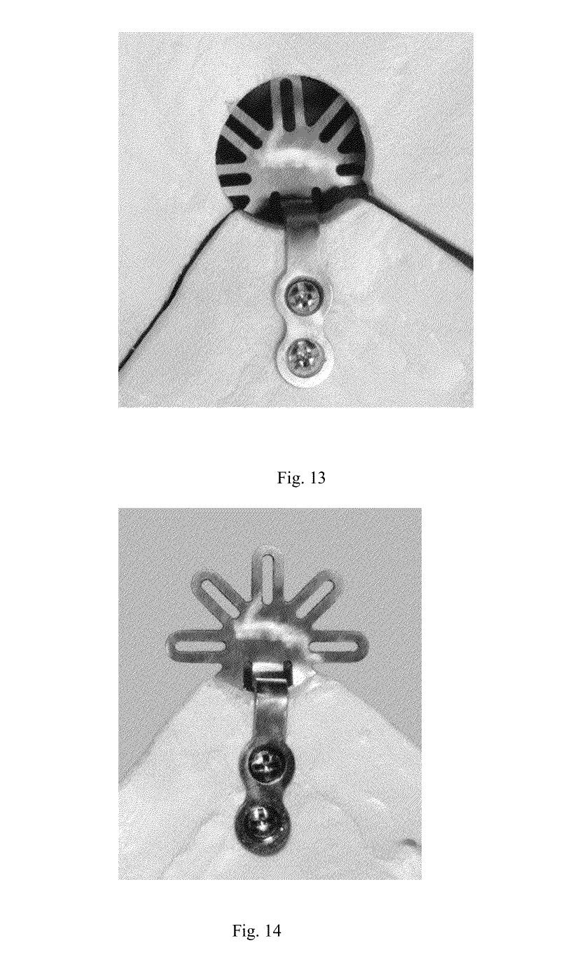

Now, reference may be made to FIG. 11 showing yet another embodiment of device of the instant invention. It comprises of one long arm (1) bent in Z-like shape provided with two holes (1′). Further, it comprises of a plurality of smaller arms (2 and 3) with hole supported on the skull as shown in FIG. 12. All the arms are integral to each other.

It requires two screws to be screwed to the skull through said holes in the longer arm. The screws are applied inside the flap as shown in FIGS. 13 and 14.

Thus, numerous embodiments can be provided relying on the concept disclosed in the invention.

It is to be noted that the present invention is susceptible to modifications, adaptations and changes by those skilled in the art. Such variant embodiments employing the concepts and features of this invention are intended to be within the scope of the present invention, which is further set forth under the following claims:

1. A medical device for anchoring and closing cranial flaps during neurosurgery, comprising:
a plurality of arms extending from a central point;
the plurality of arms comprising at least one long arm and a plurality of short arms; and
wherein the plurality of arms are made of a biocompatible material.

2. The medical device of claim 1, wherein the plurality of arms comprises two long arms disposed opposite to each other.

3. The medical device of claim 1, wherein the plurality of arms comprises
(a) two arms opposite to each other which are bent; and
(b) cross arms with a hole, wherein the cross arms are disposed between the long and short arms.

4. The medical device of claim 1, wherein the long arm comprises a bend and a hole.

5. The medical device of claim 4, wherein each arm of the plurality of short arms have a hole.

6. The medical device of claim 2, wherein a first long arm is bent and a second long arm is straight.

7. The medical device of claim 6, further comprising two screws.

8. The medical device of claim 1, wherein the long arm is bent and has two holes.

9. (canceled)

10. The medical device of claim 2, wherein each of the two long arms have a bend.

12. The medical device of claim 6, wherein the plurality of short arms each have a hole at an end, and the plurality of short arms are disposed between the long arms.

13. The medical device of claim 8, wherein each of the plurality of short arms have a hole at the end.
